(19) United States
(12) Patent Application Publication
(10) Pub. No.: US 2008/0193477 A1
(43) Pub. Date: Aug. 14, 2008

(54) Vaccination Against Dengue Virus Infection

(75) Inventors: Thomas P. Monath, Harvard, MA (US); Farshad Guirakhoo, Chaponost (FR); Niranjan Kanesa-thasan, Lexington, MA (US); Thomas H. Ermak, Brookline, MA (US); Jean Lang, Mions (FR); Remi Forrat, Serezin Du Rhone (FR)

Correspondence Address: CLARK & ELBING LLP, 101 FEDERAL STREET, BOSTON, MA, 02110, US

(73) Assignees: Acambis Inc., Cambridge, MA (US); Sanofi Pasteur SA, Lyon Cedex 07 (FR)

(21) Appl. No.: 12/063,365

(22) PCT Filed: Aug. 9, 2006
(86) PCT No.: PCT/US06/30846

§ 371 Date: Apr. 10, 2008

Related U.S. Application Data
(60) Provisional application No. 60/707,038, filed on Aug. 10, 2005.
(60) Provisional application No. 60/719,448, filed on Sep. 22, 2005.

(51) Int. Cl. A61K 39/295; A61K 39/12; A61P 31/14

(52) U.S. Cl. 424/199.1; 424/202.1

(57) ABSTRACT

This invention relates to methods and kits for use in vaccination against dengue virus infection.

### BACKGROUND OF THE INVENTION

This invention relates to vaccination against dengue virus infection.

Dengue, a disease caused by four distinct species of dengue virus (named as serotypes 1-4), is the most important vector-borne disease of humankind. Approximately 100 million persons are affected by dengue viruses annually in tropical and subtropical regions of the world (Halstead, “Epidemiology of Dengue and Dengue Hemorrhagic Fever,” CABI Publ., New York, pp. 23-44, 1997; Gubler, “Dengue and Dengue Hemorrhagic Fever,” CABI Publ., New York, pp. 1-22, 1997). A severe and potentially lethal form of disease caused by dengue virus infection, dengue hemorrhagic fever (DHF), is increasing in geographic distribution and incidence. These facts have spurred intensive efforts to construct safe and effective dengue vaccines but, despite many efforts, spanning more than 50 years, no commercially available vaccine against dengue has been developed. The development of a vaccine against dengue is thus considered to be a high priority by the World Health Organization (Chambers et al., Vaccine 15:1494-1502, 1997).

The pathogenesis of DHF drives the design of dengue vaccines. DHF is an immunopathological disease, which occurs primarily in individuals who have sustained a prior infection with one dengue serotype and then are exposed to a second, different (heterologous) serotype. Infection with any one of the four serotypes of dengue provides durable immunity to that homologous serotype, based on neutralizing antibodies. However, immunity to other, heterologous dengue serotypes following infection with one dengue serotype is of short duration, if it occurs at all (Sabin, Am. J. Trop. Med. Hyg. 1:30-50, 1952). Typically, after a few weeks or months, only binding and not neutralizing antibodies to heterologous serotypes are present. These binding but non-neutralizing antibodies may enhance subsequent infection with a heterologous dengue virus serotype, increasing the risk of severe disease (Rothman et al., Virology 257:1-6, 1999).

Given the immunopathogenesis of DHF, a successful vaccine against dengue must be safe and induce long-lasting, cross-neutralizing antibody responses against all 4 dengue virus serotypes simultaneously, so that titers do not fall to levels that would leave a subject not protected against future infection. Historically, empirical efforts to develop live, attenuated vaccine candidates have demonstrated that it is difficult to achieve a balance between sufficient attenuation (safety) and immunogenicity of candidate vaccine viruses. It has been also difficult to combine vaccine strains representing all four serotypes into an effective tetravalent mixture and a multiple dose schedule was necessary to reach seroconversion against all serotypes, with the undesirable effect of providing gaps in the immunization schedule where subjects might be sensitized to immunopathological events. Indeed, attempts to immunize with mixtures of monovalent, live dengue vaccines demonstrated significant interactions between the four virus strains and have resulted in viral interference effects (reviewed in Saluzzo, Adv. Virus Res. 61:420-444, 2003).

Genetically engineered chimeric flavivirus-based vaccines against dengue viruses have been developed, in which two sequences (i.e., sequences encoding the pre-membrane (prM) and envelope (A) proteins) of dengue serotypes 1, 2, 3, or 4 are inserted into a full-length infectious clone of yellow fever 17D virus, in place of the sequences encoding the corresponding yellow fever virus proteins (see, e.g., Guirakhoo et al., J. Virol. 75:7290-7304, 2001; Guirakhoo et al., Virology 298:146-159, 2002). These viruses are highly effective in inducing immune responses when injected into monkeys. However, preliminary data showed also some viral interference effects in human beings, which can limit immunization against all four serotypes after one dose of dengue vaccine.

The present inventors have found out a new and safe method of immunization against dengue diseases, which allows induction of a long-lasting, cross-neutralizing antibody response against dengue serotypes 1-4, while avoiding the need of a multidose dengue vaccination schedule and the potential risk associated with a primary unbalanced immune response. The method of the present invention, which uses an immunization regimen comprising the administration of a first yellow fever vaccine followed by the administration of a chimeric flavivirus-based dengue vaccine, allows the induction of a cross-neutralizing immune response against dengue viruses, which presents the advantages of appearing early (within 30 days) after the administration of the dengue vaccine, being long-lasting, and being cross-reactive against the four serotypes. Furthermore, the method of the present invention presents the additional benefit of inducing a protective immune response against yellow fever.

Price et al. (Am. J. Epid. 88:392-397, 1968) previously described a method for sequential flavivirus immunization comprising a series of three immunizations with dengue type 2 and two heterologous viruses (yellow fever and Japanese encephalitis). Furthermore, unlike the present invention, the sequence of yellow fever followed by dengue 2, without the addition of JE immunization, failed to confer cross-protective immunity.

Scott et al. (J. Infect. Dis. 148:1055-1060, 1983) showed that subjects who were previously immunized with yellow fever and subsequently inoculated with a live, attenuated dengue type 2 vaccine had enhanced immune responses to dengue type 2, which were also more durable (lasting 3 years) than in subjects without previous yellow fever immunity. The enhanced response might have been due to enhancing (binding, non-neutralizing) antibodies elicited to dengue type 2 virus by the preceding yellow fever vaccination (Eckels et al., J. Immunol. 135(6):4201-4203, 1985). However, Scott et al. did not show that yellow fever followed by dengue 2 vaccines elicited a long-lasting immune response to the other three dengue serotypes (types 1, 3, or 4). Unlike the present invention, the sequence of yellow fever followed by dengue type 2 was not shown to elicit a broad response by the neutralization test, which is the only test that predicts protective immunity.

In a recent paper, Kanesa-thasan et al. (Am. J. Trop. Med. Hyg. 69(Suppl 6):32-38, 2003) discovered boosted heterologous responses and anti-dengue antibody titers in subjects remotely vaccinated with YF following vaccination with attenuated dengue vaccines. These short-term (to day 30) antibody responses were demonstrated with antibody assays including neutralization, but the authors concluded that evidence for protection against subsequent dengue infection was inconclusive. Unlike the present invention, the authors could not demonstrate conclusively the prior timing or receipt of YF vaccination, long-term broad neutralization antibody responses, or provide evidence for cross-reactive T cell responses to dengue.

The present inventors demonstrated for the first time that induction of cross-neutralizing immunity against multiple dengue serotypes in humans may indeed be conferred by sequential administration of yellow fever and dengue chimeric viruses.

### SUMMARY OF THE INVENTION

The present invention provides a method of inducing a long-lasting, cross-neutralizing immune response to dengue virus in a patient, comprising administering to the patient:

(i) one dose of a yellow fever virus vaccine, and

(ii) one dose of a chimeric flavivirus vaccine comprising at least one chimeric flavivirus comprising a yellow fever virus backbone in which the sequence encoding envelope protein of the yellow fever virus have been replaced with a sequence encoding the envelope protein of a dengue virus, wherein the chimeric flavivirus vaccine is administered at least 30 days and up to 10 years after administration of the yellow fever vaccine. In one example, the dengue envelope sequence is a shuffled sequence.

According to one embodiment, the chimeric flavivirus comprises a yellow fever virus backbone in which the sequences encoding the membrane and envelope proteins of the yellow fever virus have been replaced with sequences encoding the membrane and envelope proteins of a dengue virus. In one example, either or both of these dengue sequences are shuffled sequences.

According to a particular embodiment, the chimeric flavivirus vaccine is administered to the patient 30, 60, or 90 days after administration of the yellow fever vaccine.

According to a particular embodiment, the chimeric flavivirus used in the dengue vaccine of the invention is composed of a yellow fever 17D (YF17D) virus backbone.

According to another embodiment, the yellow fever virus vaccine used in the method of the invention comprises a YF17D strain.

According to another embodiment, the chimeric flavivirus vaccine used in the method of the invention comprises one chimeric flavivirus comprising a yellow fever virus backbone in which the sequences encoding the membrane and envelope proteins of the yellow fever virus have been replaced with sequences encoding the membrane and envelope proteins of a dengue serotype 1 virus.

According to another embodiment, the chimeric flavivirus vaccine used in the method of the invention comprises one chimeric flavivirus comprising a yellow fever virus backbone in which the sequences encoding the membrane and envelope proteins of the yellow fever virus have been replaced with sequences encoding the membrane and envelope proteins of a dengue serotype 2 virus.

According to another embodiment, the chimeric flavivirus vaccine used in the method of the invention comprises one chimeric flavivirus comprising a yellow fever virus backbone in which the sequences encoding the membrane and envelope proteins of the yellow fever virus have been replaced with sequences encoding the membrane and envelope proteins of a dengue serotype 3 virus.

According to another embodiment, the chimeric flavivirus vaccine used in the method of the invention comprises one chimeric flavivirus comprising a yellow fever virus backbone in which the sequences encoding the membrane and envelope proteins of the yellow fever virus have been replaced with sequences encoding the membrane and envelope proteins of a dengue serotype 4 virus.

According to a particular embodiment, the chimeric flavivirus vaccine used in the method of the invention is a monovalent vaccine or a tetravalent vaccine.

According to another embodiment, the method of the invention further comprises the administration of a booster dose of the above-defined chimeric flavivirus vaccine, 6 months to 10 years after the first dose of the chimeric flavivirus vaccine.

According to another aspect, the present invention concerns a kit comprising:

(i) a yellow fever virus vaccine, and

(ii) a chimeric flavivirus vaccine comprising at least one chimeric flavivirus comprising a yellow fever virus backbone in which the sequence encoding the envelope protein of the yellow fever virus has been replaced with the sequence encoding the envelope protein of a dengue virus. In one example, the dengue envelope sequence is a shuffled sequence.

According to one embodiment, the chimeric flavivirus comprises a yellow fever virus backbone in which the sequences encoding the membrane and envelope proteins of the yellow fever virus have been replaced with sequences encoding the membrane and envelope proteins of a dengue virus. In one example, either one or both of these dengue sequences are shuffled sequences.

According to one embodiment of the kit of the invention, the yellow fever virus vaccine comprises a YF17D strain, wherein YF17D comprises a number of substrains used for vaccination against yellow fever (including 17D-204, 17D-213, and 17DD).

According to another embodiment, the chimeric flavivirus is composed of a YF17D virus backbone.

According to another embodiment of the kit of the invention, the chimeric flavivirus vaccine comprises one chimeric flavivirus comprising a YF17D virus backbone in which the sequences encoding the membrane and envelope proteins of the yellow fever virus have been replaced with sequences encoding the membrane and envelope proteins of a dengue serotype 1 virus.

According to another embodiment of the kit of the invention, the chimeric flavivirus vaccine comprises one chimeric flavivirus comprising a yellow fever virus backbone in which the sequences encoding the membrane and envelope proteins of the yellow YF17D virus have been replaced with sequences encoding the membrane and envelope proteins of a dengue serotype 2 virus.

According to another embodiment of the kit of the invention, the chimeric flavivirus vaccine comprises one chimeric flavivirus comprising a yellow fever virus backbone in which the sequences encoding the membrane and envelope proteins of the YF17D virus have been replaced with sequences encoding the membrane and envelope proteins of a dengue serotype 3 virus.

According to another embodiment of the kit of the invention, the chimeric flavivirus vaccine comprises one chimeric flavivirus comprising a YF17D virus backbone in which the sequences encoding the membrane and envelope proteins of the yellow fever virus have been replaced with sequences encoding the membrane and envelope proteins of a dengue serotype 4 virus.

According to another embodiment the kit as defined above further comprises at least one booster dose of a chimeric flavivirus vaccine as defined above.

According to another embodiment, the invention concerns the use of the viruses noted above and elsewhere herein in the prevention and treatment of dengue virus infection, as well as the use of these viruses in the preparation of medicaments for this purpose.

### DEFINITIONS

By “cross-neutralizing immune response” we mean a specific immune response comprising neutralizing antibodies against multiple (up to 4) different dengue serotypes. Induction of a cross-neutralizing immune response can be easily determined by a reference plaque reduction neutralization assay (PRNT₅₀). For example, induction of a cross-neutralizing immune response can be determined by one of the PRNT₅₀ assays as described in Example 1. A serum sample is considered to be positive for the presence of cross-neutralizing antibodies when the neutralizing antibody titer thus determined is at least superior or equal to 1:10 in at least one of these assays.

By “long-lasting immune response” we mean a positive cross-neutralizing immune response as defined above, which can be detected in human serum at least 6 months, advantageously, at least 12 months after the administration of a chimeric flavivirus vaccine as defined below.

By “patient” we mean yellow fever-naïve individuals including adults and children.

By “yellow fever naïve” individuals we mean individuals with no documented vaccination against yellow fever for more than 10 years and/or no certified yellow fever virus infection for more than 10 years.

By “yellow fever immune individuals” we thus mean, within the framework of the present invention, individuals with a documented vaccination against yellow fever and/or with a certified yellow fever virus infection that has occurred 10 years ago or less, e.g., 5 years or less, e.g., 4, 3, 2, or 1 years ago, or even 6, 5, 4, 3, or 2 months ago, and in any case more than 30 days ago.

By “chimeric flavivirus” we mean a chimeric flavivirus composed of a yellow fever virus backbone in which the sequence encoding the envelope protein of the yellow fever virus has been replaced with the sequence encoding the envelope protein of a dengue virus. Advantageously, a chimeric flavivirus is composed of a yellow fever virus backbone in which the sequences encoding the membrane and envelope proteins of the yellow fever virus have been replaced with the sequences encoding the membrane and envelope proteins of a dengue virus. The yellow fever backbone can advantageously be from a vaccine strain, such as YF17D or YF17DD. These chimeric flaviviruses are defined in more detail below and are named YF/dengue-N, with N identifying the dengue serotype.

By “chimeric flavivirus vaccine” we mean an immunogenic composition comprising an immunoeffective amount at least one chimeric flavivirus as defined above and a pharmaceutically acceptable excipient.

The chimeric flavivirus vaccine is said to be “monovalent” when the vaccine comprises one chimeric flavivirus expressing protein(s) of one dengue serotype. Examples of monovalent vaccines are vaccines comprising YF/dengue-1, YF/dengue-2, YF/dengue-3, or YF/dengue-4, advantageously YF/dengue-2.

The chimeric flavivirus vaccine is said to be “bivalent” when the vaccine comprises chimeric flaviviruse(s) expressing protein(s) of two different dengue serotypes. Examples of bivalent vaccines are vaccines comprising YF/dengue-2 and YF/dengue-4, or YF/dengue-2 and YF/dengue-3, or YF/dengue-2 and YF/dengue-1.

The chimeric flavivirus vaccine is said to be “trivalent” when the vaccine comprises chimeric flaviviruse(s) expressing protein(s) of three different dengue serotypes. Examples of trivalent vaccines are vaccines comprising YF/dengue-2, YF/dengue-1, and YF/dengue-4, or YF/dengue-2, YF/dengue-3, and YF/dengue-4.

The chimeric flavivirus vaccine is said to be “tetravalent” when the vaccine comprises chimeric flaviviruse(s) expressing protein(s) of four different dengue serotypes. An example of a tetravalent vaccine is a vaccine that includes YF/dengue-1, YF/dengue-2, YF/dengue-3, and YF/dengue-4.

By “immunoeffective amount of a chimeric flavivirus” we mean an amount of chimeric flavivirus capable of inducing, after administration in a yellow fever immune individual, a cross-neutralizing immune response as defined above. Typically, an immunoeffective amount of a chimeric flavivirus is comprised of between 10² and 10⁷, e.g., between 10³ and 10⁶, such as an amount of 10⁴, 10⁵, or 10⁶, infectious units (e.g., plaque-forming units or tissue culture infectious doses) per serotype, per dose.

A central advantage of the method of the present invention is the ability to induce neutralizing antibodies against all four dengue serotypes quickly and simultaneously, thereby protecting against dengue fever and thus avoiding the potential associated risks of developing dengue hemorrhagic fever on subsequent natural exposure to dengue infection. Neutralizing antibodies directed against the dengue envelope protein are considered the principal mediator of protective immunity against infection, therefore the demonstration of neutralizing antibodies is considered as a relevant surrogate of a neutralizing immunity in patients.

Other features and advantages of the invention will be apparent from the following detailed description, the claims, and the drawings.

### BRIEF DESCRIPTION OF THE DRAWINGS

FIG. 1 is a graph showing IFNγ responses to vaccine (study Day 31 minus Day 1). The two doses of ChimeriVax™-Den2 gave equivalent T cell responses. The response was not inhibited in subjects previously vaccinated with yellow fever virus vaccine.

### DETAILED DESCRIPTION

The invention provides a method for inducing in a patient long-lasting, cross-neutralizing immunity to all four dengue serotypes (1-4) using a simple, two-step procedure. The targeted population is thus composed especially of the following patients at risk of dengue infection: foreign travelers, expatriate and military personnel, as well as inhabitants of regions in which dengue is endemic. In this method, a patient is first immunized with a dose (preferably one dose, but possibly more than one dose (e.g., 2 or 3 doses)) of a yellow fever virus vaccine (e.g., a commercially available, live attenuated vaccine; see below). After an appropriate time interval of at least 30 days, which allows in particular for quiescence of the innate immune response induced by the yellow fever virus vaccine, the second step of the method is carried out, which involves administration of one dose of a chimeric flavivirus vaccine comprising one or more live, attenuated chimeric viruses, each comprising a yellow fever virus backbone in which one or more sequences encoding structural proteins (e.g., pre-membrane and envelope proteins) have been replaced with the sequences encoding the corresponding proteins of a dengue virus (e.g., dengue 1, 2, 3, or 4). The present inventors have shown that this sequence of immunization elicits high neutralizing antibody titers against all four dengue serotypes. These antibodies persisted at high levels over 6 months and even over 12 months after the dengue vaccine administration, indicating that broad dengue immunity was long-lasting. Since the initial immunizing/priming agent (yellow fever vaccine) is incapable of sensitizing the subject to DHF, there was no danger that the first, priming inoculation would leave the subject vulnerable to this disease if the second injection was delayed or not performed. These results were unexpected, as even sequential infection with two dengue virus serotypes, which are much more closely related to one another based on genome sequence and antigenic relationships than yellow fever is related to dengue, does not induce solid protection or broad cross-neutralizing antibody responses against infection with the remaining two dengue serotypes. Further demonstration of the unexpected nature of the method of sequential vaccination of the invention was provided by an examination of the yellow fever antibody response following the second step (inoculation of the chimeric dengue virus). The method of the invention is described further, as follows.

### Yellow Fever Virus Vaccines

As is noted above, the first step of the method of the invention involves administration to a patient of one dose of a yellow fever virus vaccine. Examples of such vaccines that can be used in the invention include live, attenuated vaccines, such as those derived from the YF17D strain, which was originally obtained by attenuation of the wild-type Asibi strain (Smithburn et al., “Yellow Fever Vaccination,” World Health Organization, p. 238, 1956; Freestone, in Plotkin et al. (eds.), Vaccines, 2^{nd} edition, W.B. Saunders, Philadelphia, U.S.A., 1995). An example of a YF17D strain from which vaccines that can be used in the invention can be derived is YF17D-204 (YF-VAX®, Sanofi-Pasteur, Swiftwater, Pa., USA; Stamaril®, Sanofi-Pasteur, Marcy-L'Etoile, France; ARILVAX™, Chiron, Speke, Liverpool, UK; FLAVIMUN®, Berna Biotech, Bern, Switzerland; YF17D-204 France (×15067, X15062); YF17D-204, 234 US (Rice et al., Science 229:726-733, 1985)), while other examples of such strains that can be used are the closely related YF17DD strain (GenBank Accession No. U 17066), YF17D-213 (GenBank Accession No. U17067), and yellow fever virus 17DD strains described by Galler et al., Vaccines 16(9/10):1024-1028, 1998. In addition to these strains, any other yellow fever virus vaccine strains found to be acceptably attenuated in humans, such as human patients, can be used in the invention.

The yellow fever virus vaccines used in the invention can be obtained from commercial sources (see above) or can be prepared using methods that are well known in the art. In one example of such methods, chicken embryos are inoculated with virus at a fixed passage level, and then virus isolated from supernatants of centrifuged homogenate is freeze-dried. In other methods, the yellow fever strain is grown in cultured chicken embryo fibroblasts (see, e.g., Freire et al., Vaccine 23(19):2501-2512, 2005) or other cultured cells for manufacture of viral vaccines such as Vero cells. The yellow fever virus vaccines are generally stored in lyophilized form prior to use. When needed for administration, the vaccines are reconstituted in an aqueous solution (typically, about 0.5 mL), such as a 0.4% sodium chloride solution, and then are administered by subcutaneous injection in, e.g., the deltoid muscle. Other modes of administration determined to be appropriate by those of skill in the art (e.g., intramuscular or intradermal injection, or percutaneous administration using methods that deliver virus to the superficial layers of the skin) can also be used. The vaccine can be administered in dosages ranging from, for example, 2-5 (e.g., 3 or 4) log₁₀ plaque-forming units (PFU) per dose. All commercialized vaccines are used according to manufacturer recommendations. In one embodiment, the first step of the method of the invention consists of the administration of one dose of Stamaril™ or of one dose of YF-VAX®.

The method of the present invention can also be adapted to be used with yellow fever immune patients. In such a case, the method only comprises the second step involving the administration of one dose of a chimeric flavivirus vaccine as defined below. The said method is also included within the scope of the present invention.

### Chimeric Flavivirus Vaccines

The second step of the method of immunization according to the invention comprises administration of one dose of a chimeric flavivirus vaccine as defined above. For the sake of clarity, in the following description, the invention is only defined in relation to the use of chimeric flaviviruses in which the chimeric flavivirus is composed of a yellow fever virus backbone in which the sequences encoding the membrane and envelope proteins of the yellow fever virus have been replaced with the sequences encoding the membrane and envelope proteins of a dengue virus. The invention also includes the use of other chimeras, such as chimeras in which only one protein (e.g., the envelope protein) of a yellow fever vaccine strain has been replaced, or chimeras in which all three structural proteins have been replaced.

Chimeric viruses that can be used in the present invention include those based on the human yellow fever vaccine strain, YF17D (e.g., YF17D-204, YF17D-213, or YF17DD), as described above. In these viruses, the pre-membrane and envelope proteins of the yellow fever virus are replaced with the pre-membrane and envelope proteins of a dengue virus (serotype 1, 2, 3, or 4). In one embodiment of the present invention, the chimeric viruses are composed of a YF17D-204 backbone in which the sequence encoding pre-membrane and envelope proteins of the yellow fever virus are replaced with the sequences encoding the pre-membrane and envelope proteins of wild type dengue serotype 1, 2, 3, and/or 4, e.g., with the sequences encoding the pre-membrane and envelope proteins of dengue 1 virus PUO-359, dengue 2 virus PUO-218, dengue 3 virus PaH-881/88, or dengue 4 virus 1228. Details of the construction of these and related chimeric virus constructs are provided, for example, in the following publications: WO 98/37911; WO 01/39802; Chambers et al., J. Virol. 73:3095-3101, 1999; WO 03/103571; WO 2004/045529; U.S. Pat. No. 6,696,281; U.S. Pat. No. 6,184,024; U.S. Pat. No. 6,676,936; U.S. Pat. No. 6,497,884; Guirakhoo et al., J. Virology 75:7290-7304, 2001; Guirakhoo et al., Virology 298:146-159, 2002; and Caufour et al., Virus Res. 79(1-2):1-14, 2001. As one specific example of a chimeric flavivirus that can be used in the invention, we make note of the following chimeric flavivirus, which was deposited with the American Type Culture Collection (ATCC) in Manassas, Va., U.S.A. under the terms of the Budapest Treaty and granted a deposit date of Jan. 6, 1998: Chimeric Yellow Fever 17D/Dengue Type 2 Virus (YF/DEN-2; ATCC accession number ATCC VR-2593).

The chimeric flaviviruses used in the methods of the invention can, optionally, include attenuating mutations in dengue virus sequences. For example, the dengue sequences can include a deletion or substitution of envelope amino acid 204 (dengue serotypes 1, 2, and 4) or 202 (dengue serotype 3), which is lysine in the wild type viruses. In one example of such a substitution, the lysine at this position is replaced with arginine. In other examples, one or more other amino acids in the region of amino acids 200-208 (or combinations of these amino acids) are mutated, with specific examples including the following: position 202 (K) of dengue-1; position 202 (E) of dengue-2; position 200 of dengue-3 (K); and positions 200 (K), 202 (K), and 203(K) of dengue-4. These residues can be substituted with, for example, arginine. These mutations are described in detail in WO 03/103571, the content of which is incorporated here by reference.

In addition to the chimeras described above, other chimeras that contain structural proteins including epitopes from more than one (2, 3, or 4) dengue virus serotype can be used in the invention. In one example, chimeras can be made using shuffling technology, which involves cycles of fragmentation, rejoining, and selection of sequences that are being shuffled (see, e.g., Locher et al., DNA Cell Biol. 24(4):256-263, 2005). Thus, in the present case, sequences encoding envelope and/or pre-membrane proteins from a desired subset of dengue serotypes (or all dengue serotypes) can be processed in this way to generate shuffled envelope and/or pre-membrane sequences, which are then used to substitute the corresponding sequences of a yellow fever virus backbone as described herein (e.g., YF17D). Such a chimeric YF/Den1-4 shufflant (assuming shuffled sequences include epitopes from all four serotypes) can be produced by, for example, transfection of Vero cells with chimeric RNA transcripts and recovery of live virus from the supernatant as described previously (Guirakhoo et al., J. Virol. 75(16):7290-7304, 2001) and mentioned elsewhere herein. These shuffled chimeras can be used in the invention in vaccination regimens involving administration of the shuffled chimera following yellow fever (e.g., YF17D) vaccination, or in any of the combination methods described elsewhere herein.

The chimeric viruses described above can be made using standard methods in the art. For example, an RNA molecule corresponding to the genome of a virus can be introduced into primary cells, chicken embryos, or diploid cell lines, from which (or the supernatants of which) progeny virus can then be purified. Other methods that can be used to produce the viruses employ heteroploid cells, such as Vero cells (Yasumura et al., Nihon Rinsho 21:1201-1215, 1963). In an example of such methods, a nucleic acid molecule (e.g., an RNA molecule) corresponding to the genome of a virus is introduced into the heteroploid cells, virus is harvested from the medium in which the cells have been cultured, harvested virus is treated with a nuclease (e.g., an endonuclease that degrades both DNA and RNA, such as Benzonase™; U.S. Pat. No. 5,173,418), the nuclease-treated virus is concentrated (e.g., by use of ultrafiltration using a filter having a molecular weight cut-off of, e.g., 500 kDa), and the concentrated virus is formulated for the purposes of vaccination. Details of this method are provided in WO 03/060088 A2, which is incorporated herein by reference. Further, methods for producing chimeric viruses are described in the documents cited above in reference to the construction of chimeric virus constructs.

Formulation of the chimeric viruses used in the methods of the invention can be carried out using methods that are standard in the art. Numerous pharmaceutically acceptable solutions for use in vaccine preparation are well known and can readily be adapted for use in the present invention by those of skill in this art (see, e.g., Remington's Pharmaceutical Sciences (18^{th} edition), ed. A. Gennaro, 1990, Mack Publishing Co., Easton, Pa.). In two specific examples, the viruses are formulated in Minimum Essential Medium Earle's Salt (MEME) containing 7.5% lactose and 2.5% human serum albumin or MEME containing 10% sorbitol. However, the chimeric flaviviruses can simply be diluted in a physiologically acceptable solution, such as sterile saline or sterile buffered saline. In another example, the viruses can be administered and formulated, for example, in the same manner as the yellow fever 17D vaccine, e.g., as a clarified suspension of infected chicken embryo tissue or a fluid harvested from cell cultures infected with a chimeric virus.

The chimeric flavivirus vaccines of the invention are classically stored either in the form of a frozen liquid composition or in the form of a lyophilized product. For that purpose, the chimeric flavivirus can be mixed with a diluent classically a buffered aqueous solution comprising cryoprotective compounds such as sugar alcohol and stabilizer. Before use, the lyophilized product is mixed with a pharmaceutically acceptable diluent or excipient such as a sterile NaCl 4% solution to reconstitute a liquid injectable chimeric flavivirus vaccine.

In the method of the invention, the chimeric flavivirus vaccine can be a monovalent, a bivalent, a trivalent, or a tetravalent vaccine.

According to one embodiment, the chimeric flavivirus vaccine is a monovalent vaccine in which the chimeric virus is composed of a YF17D-204 backbone in which the sequence encoding pre-membrane and envelope proteins of the yellow fever virus are replaced with the sequences encoding the pre-membrane and envelope proteins of dengue 2 virus PUO-218.

According to another embodiment, the chimeric flavivirus vaccine is a tetravalent vaccine i.e. a vaccine comprising chimeric virus(es) expressing antigen(s) from the four dengue (1 to 4) virus serotypes. In a particular embodiment, this tetravalent vaccine includes advantageously four chimeric flaviviruses composed respectively of a YF17D-204 backbone in which the sequences encoding pre-membrane and envelope proteins of the yellow fever virus are replaced with sequences encoding the pre-membrane and envelope proteins of dengue 1 virus PUO-359 (YF/dengue1), dengue 2 virus PUO-218 (YF/dengue2), dengue 3 virus PaH-881/88 (YF/dengue3), or dengue 4 virus 1228 (YF/dengue4). This specific tetravalent vaccine is named in Example 2 below ChimeriVax™-DEN tetravalent.

Examples of tetravalent chimeric flavivirus vaccines appropriate to be used in the method of the invention are also described in detail in WO 03/101397, the content of which is integrated herein by reference. Multivalent vaccines may be obtained by combining individual monovalent dengue vaccines.

The chimeric viruses of the invention can be administered using methods that are well known in the art. For example, the viruses can be formulated as sterile aqueous solutions containing between 10² and 10⁷, e.g., containing between 10³ and 10⁶, such as 10⁴, 10⁵, or 10⁶ infectious units (e.g., plaque-forming units or tissue culture infectious doses) per serotype in a dose volume of 0.1 to 1.0 mL, to be administered by, for example, subcutaneous, intramuscular, or intradermal routes. In one embodiment, the chimeric flavivirus vaccine is a monovalent, bivalent, trivalent, or tetravalent vaccine comprising advantageously 10⁵ pfu per serotype, per dose, and is administered subcutaneously. In addition, because flaviviruses may be capable of infecting the human host via mucosal routes, such as the oral route (Gresikova et al., “Tick-borne Encephalitis,” In The Arboviruses, Ecology and Epidemiology, Monath (ed.), CRC Press, Boca Raton, Fla., 1988, Volume IV, 177-203), an administration by mucosal (e.g., oral) routes could also be contemplated.

Optionally, adjuvants that are known to those skilled in the art can be used in the administration of the viruses used in the invention. Adjuvants that can be used to enhance the immunogenicity of the chimeric flaviviruses include, for example, agonists and antagonists of toll-like receptors (TLRs).

### Immunization Methods

As is noted above, the invention generally involves administration of a yellow fever vaccine strain (e.g., a YF17D strain, as is noted above), followed by administration of one or more chimeric flaviviruses, in each of which the pre-membrane and envelope proteins of the yellow fever virus have been replaced with the corresponding proteins of a dengue virus (serotype 1, 2, 3, or 4). The yellow fever virus vaccine is administered using standard methods (e.g., by subcutaneous, intramuscular, or intradermal injection, or by percutaneous administration employing a device that delivers virus to the superficial skin), in amounts ranging from, for example, 2-5 (e.g., 3 or 4) log₁₀ plaque forming units (PFU) per dose, which typically is in a volume of about 0.5 mL for subcutaneous injection, 0.1 mL for intradermal injection, or 0.002-0.02 mL for percutaneous administration.

To allow a sufficient time for quiescence of the innate immune response induced by the yellow fever vaccine, the chimeric flavivirus vaccine is administered at least between 30 days and 10 years, in particular between 30 days and 5 years, such as between 30 days and 1 to 3 years, advantageously, 30, 60, or 90 days, after the yellow fever vaccine, using standard methods and in amounts ranging from, 10² and 10⁷, e.g., from 10³ and 10⁶, such as 10⁴, 10⁵, or 10⁶ infectious units (expressed as pfu or tissue culture infection doses) per serotype per dose. Further, in the case of administration of bi-, tri-, or tetravalent formulations (see below), in general, the amounts of each chimera in such a vaccine are equivalent, although use of differing amounts of each chimera is also included in the invention.

The methods of the invention can thus involve, for example, administration of a yellow fever virus vaccine on Day 0 and administration of a YF/dengue-1, YF/dengue-2, YF/dengue-3, and/or YF/dengue-4 chimera on Day 30 (or at a later time, as noted above). The chimera can be administered as a monovalent vaccine (i.e., a vaccine including only one of the following chimeric virus: YF/dengue-1, YF/dengue-2, YF/dengue-3, or YF/dengue-4), a bivalent formulation (e.g., a vaccine including two of the chimeras listed above, e.g., including advantageously YF/dengue-2 and YF/dengue-4, or YF/dengue-2 and YF/dengue-3, or YF/dengue-2 and YF/dengue-1), a trivalent vaccine (e.g., a vaccine including three of the chimeras listed above, advantageously, a vaccine comprising YF/dengue-2, YF/dengue-1, and YF/dengue-4, or YF/dengue-2, YF/dengue-3, and YF/dengue-4), or a tetravalent vaccine.

The method of the invention leads to a seroconversion (i.e., induction of a neutralizing immune response) for four dengue serotypes after only one dose of the chimeric flavivirus vaccine. Although additional doses of the chimeric flavivirus vaccine are not needed to reach the desired seroconversion and long-lasting, cross-neutralizing immune response, administration of booster doses of the chimeric flavivirus vaccine are contemplated in the present invention. The booster dose(s) of the chimeric vaccine of the invention may be needed to sustain the cross-neutralizing immune response for a longer period of time and can be administered between 6 months and 5 to 10 years after the first chimeric dengue vaccine dose, e.g., 6 months, 1 year, 2 years, 3 years, 4 years, or 5 years, or even 10 years after the first chimeric flavivirus vaccine dose. The booster chimeric flavivirus vaccine can be different from or advantageously identical to the first chimeric flavivirus vaccine administered. The description given above in relation to the chimeric flavivirus vaccine to be administered in the method of the invention applies mutatis mutandis to the chimeric flavivirus vaccine booster. The booster can thus be a monovalent, bivalent, trivalent, or tetravalent vaccine, with respect to the dengue serotypes present in the vaccine. Thus, an example of a method of the invention may involve administration of one dose of a yellow fever vaccine, followed by one dose of a monovalent chimeric flavivirus vaccine (dengue 1, 2, 3, or 4, advantageously, dengue 2), which is then followed by administration of (i) a monovalent chimeric flavivirus vaccine of the same or different serotype as the initially administered chimera (advantageously of serotype 4), (ii) a bivalent chimeric flavivirus vaccine, which may or may not include the same serotype as the initial chimera (e.g., advantageously dengue 1 and 2 followed by dengue 3 and 4), (iii) a trivalent chimeric flavivirus vaccine, which may or may not include the same serotype as the initial chimera, or (iv) a tetravalent chimeric flavivirus vaccine. The booster chimeric flavivirus vaccine is advantageously identical to the first chimeric flavivirus vaccine in regard to its antigenic composition.

The invention thus also concerns a composition for inducing in a patient a long-lasting, cross-neutralizing immune response to dengue virus including (i) a yellow fever virus vaccine and (ii) a chimeric flavivirus vaccine for a sequential administration, in which the chimeric yellow fever vaccine is administered at least 30 days and up to 10 years after administration of the yellow fever virus vaccine.

The invention also includes kits that include a yellow fever virus vaccine and/or one or more chimeric flavivirus vaccine(s), as described herein. The kits of the invention can also include instructions for using the kits in the vaccination methods described herein. These instructions can include, for example, indications as to the amounts of vaccine to administer and/or information as to when the vaccines are to be administered.

All publications and patent applications cited in this specification are herein incorporated by reference as if each individual publication or patent application were specifically and individually indicated to be incorporated by reference.

The invention is based, in part, on the experimental results described in the following Examples.

### EXAMPLES

In the Examples set forth below, experiments and clinical studies are described which show the effects of prior immunity to yellow fever virus on subsequent vaccination with a chimeric YF/dengue-2 vaccine (Example 1) or a tetravalent (YF/dengue-1, YF/dengue-2, YF/dengue-3, and YF/dengue-4) vaccine (Example 2). These studies include analysis of neutralizing antibodies, seroconversion, viremia, and T cell responses.

### Example 1

### ChimeriVax™-Den2

Commercial YF17D vaccine (YF-VAX®) was purchased from Aventis-Pasteur, Swiftwater, Pa. ChimeriVax™-DEN2 is a live, attenuated, genetically engineered virus in which the sequences encoding two structural proteins (prM and E) of YF17D vaccine virus are replaced with the corresponding sequences of the DEN2 virus (strain PUO-218 isolated from a case of classical dengue fever, Bangkok, Thailand). The genetic construction of a chimeric viral genome is accomplished using circular cloned deoxyribonucleic acid (cDNA). Full-length cDNA is transcribed to ribonucleic acid (RNA) and the RNA used to transfect cell cultures, which produce live virus (Guirakhoo et al., J. Virol. 75:7290-7304, 2001).

The vaccine virus was produced according to current Good Manufacturing Practice (cGMP). The virus is grown in Vero (African green monkey kidney) cells from cell banks that have been tested for adventitious agents, according to Food and Drug Administration (FDA) guidelines for mammalian cell culture derived products. Supernatant fluid from Vero cell cultures containing vaccine virus is harvested, clarified from cellular debris by filtration, and treated with a nuclease (Benzonase®) to digest nucleic acid molecules derived from host cells. The nuclease-treated bulk virus is then concentrated by ultrafiltration and purified by diafiltration. The vaccine is formulated with Human Serum Albumin (HSA) USP (2.5%) and lactose USP (7.5%). The vaccine was shown to be sterile and free of mycoplasma, retroviruses [by Product Enhanced Reverse Transcriptase (PERT)], and adventitious viruses by in vitro and in vivo tests. The final vial of vaccine was tested for sterility, potency, identity, pH, appearance, osmolarity, HSA, lactose, endotoxin, safety (modified general safety in mice and guinea pigs), and mouse neurovirulence.

Preclinical studies in monkeys showed that ChimeriVax™-DEN2 is highly immunogenic and well tolerated afterinoculation of doses ranging from 2 to 5 log₁₀ PFU (Guirakhoo et al., J. Virol. 74(12):5477-5485, 2000). A low-grade viremia occurred during the first week after vaccination in monkeys, similar to that induced by yellow fever 17D vaccine. A single subcutaneous injection of 2 log₁₀ PFU vaccine (the minimum tested dose) induced neutralizing antibodies after 15-30 days, which protected against challenge with wild type DEN2 virus.

Clinical Study with Monovalent ChimeriVax™-DEN2 Vaccine

A randomized, double-blind, single-center outpatient study was performed. After screening, eligible yellow fever (YF) naïve subjects were randomized to a single vaccination with low or high dose ChimeriVax™-DEN2 (3.0 or 5.0 log₁₀ plaque forming units) or YF-VAX®. There was also an open component in which antibody response to high dose ChimeriVax™-DEN2 vaccination was evaluated in YF-immune subjects. Subjects were followed-up at Days 1-11, 21, and 31 for antibody response and safety assessments, and the durability of antibody response was assessed at 6 and 12 months post-vaccination.

After screening, 42 eligible YF-naïve subjects were randomized equally into 3 groups (high or low dose ChimeriVax™-DEN2 or YF-VAX®). On Day 1, 14 subjects received a single subcutaneous (SC) vaccination with ChimeriVax™-DEN2 (high or low dose) or YF-VAX®. An additional 14 subjects who were immune to YF (from previous YF vaccination done 6 months to 5 years before the chimeric dengue vaccine administration) received high dose ChimeriVax™-DEN2. Subjects returned to the clinic on Days 2-11, 21, and 31. Safety assessments were performed at specified time points during Days 1-31. Antibody responses to homologous vaccine strains and wild-type strains of DEN2, and neutralizing antibodies to YF17D and prototype strains of DEN1-4, were measured on Days 1 (pre-vaccination) and 31. The study was un-blinded after completion of the treatment period, and subjects were assessed at 6 and 12 months post-vaccination for the durability of the antibody response.

The proportion of subjects who developed neutralizing antibodies at a level >1:10 to different strains representing the four dengue serotypes was determined. The effect of prior immunization with YF on the DEN2 seroconversion rate in the YF-immune and YF-naïve groups receiving high dose ChimeriVax™-DEN2 was analyzed. The geometric mean neutralizing antibody titers in each treatment group and to all four dengue serotypes were measured at various time intervals after vaccination, up to 12 months.

### Viremia

Virus circulating in the blood (viremia) is a measure of replication of the different live, attenuated vaccines used in the study. Viremia was assayed by a plaque assay in Vero cells. The number of subjects who developed viremia in the 11 days after vaccination is shown by day of visit in Table 1. More YF-naïve subjects vaccinated with ChimeriVax™-DEN2 than YF-VAX® developed viremia on one or more study days: 8 (57%) in the ChimeriVax™-DEN2 5.0 log₁₀ PFU group and 9 (64%) in the ChimeriVax™-DEN2 3.0 log₁₀ PFU group, compared with 2 (14%) in the YF-VAX® group. Slightly higher numbers of YF-immune subjects, compared with YF-naïve subjects, developed viremia following vaccination with ChimeriVax™-DEN2 5.0 log₁₀ PFU (11/14 [79%] in YF-immune subjects vs. 8/14 [57%] in YF-naïve subjects), but the difference was not statistically significant (p=0.4724). Most subjects developed viremia between days 5-7. The quantitative viremia measures (mean peak, mean duration, AUC) were also higher in the YF immune group, but again the differences were not statistically significant and importantly no impact on the safety profile was observed.

TABLE 1
Viremia summary measures

| | YF-VAX ® 5.04 | ChimeriVax ™- | ChimeriVax ™- | ChimeriVax ™- | |
|---|---|---|---|---|---|
| | log₁₀ PFU YF- | DEN2 3.0 log₁₀ | DEN2 5.0 log₁₀ | DEN2 5.0 log₁₀ | |
| Treatment Group | naïve | PFU YF-naïve | PFU YF-naïve | PFU YF-immune | P-value^{c} |
| No. subjects | 14 | 14 | 14 | 14 | |
| No. (%) subjects | 2 (14) | 9 (64) | 8 (57) | 11 (79) | 0.4724 |
| viremic | | | | | |
| Peak (PFU^{a}/mL) [SD] | 20.0 [51.44] | 11.4 [12.31] | 12.1 [16.72] | 29.3 [38.72] | 0.1484 |
| Duration (Days) [SD] | 0.4 [1.16] | 1.2 [1.42] | 1.4 [1.65] | 1.9 [1.23] | 0.2357 |
| AUC^{b} (PFU/mL) [SD] | 44.3 [116.86] | 20.0 [33.74] | 20.7 [32.04] | 50.4 [67.61] | 0.0908 |

^{a}PFU = plaque-forming units, measured in Vero cell cultures
^{b}Area under the curve
^{c}Pairwise comparison of ChimeriVax ™-DEN2 5.0 log₁₀ in YF naïve versus immune subjects

### Neutralizing Antibodies

Three different wild-type strains of dengue type 2 (16681, JAH, and PR-159), as well as the homologous vaccine strain (ChimeriVax™-DEN2), were used in neutralization tests. Wild-type strains of heterologous dengue serotypes 1 (16007), 3 (16562), and 4 (1036) were also used to measure the breadth of the neutralizing antibody response. The proportion of subjects seroconverting (demonstrating a neutralizing antibody titer at least superior or equal to 1:10 between Day 1 and Day 30) was determined. In addition, the geometric mean neutralizing antibody titers were measured.

The Plaque Reduction Neutralization Test (PRNT₅₀) used for CVD2, PR-159, and JAH comprises the following steps:

Heat-inactivated serum was serially diluted two-fold and mixed with an equal volume of virus to achieve 30-50 pfu/well. The serum-virus mixtures were incubated at 4° C. for 18+/−2 hours, then added to Vero cell monolayers in 12-well culture plates. After a 60+/−10 minute incubation, the monolayers were overlaid with 0.84% carboxymethylcellulose in growth medium. Plates were then incubated at 37° C. under 5% CO₂ for 3-5 days.

Monolayers were fixed with 7.4% formalin, then blocked and permeabilized with 2.5% non-fat dry milk in PBS-Tween20 plus 0.5% Triton X-100. Anti-Dengue 2 primary antibody (3H5, 1:5000) was incubated 60+/−10 minutes, followed by goat anti-mouse IgG alkaline phosphatase (1:500). After 60+/−10 minutes incubation, substrate, BCIP-NBT containing 0.36 mM levamisole was added. The reaction was stopped after sufficient staining had occurred.

Plaques were counted and PRNT₅₀ titers determined. PRNT₅₀ titers were defined as the first serum dilution in which the plaque count is equal to or less than 50% of the negative control plaque count. A serum is considered to be positive for the presence of neutralizing antibodies when the neutralizing antibody titer thus determined is at least superior or equal to 1:10.

For the other strains, the PRNT₅₀ assay has been carried out in another laboratory according to the following protocol described by Russell et al. (J. Immunol. 99:285-290, 1967). Plaque count was determined by using the LLC-MK2 plaque assay single overlay technique. Sera are thawed, diluted, and heat-inactivated by incubation at 56° C. for 30 minutes. Serial, 4-fold-dilutions of serum are made (1:5, 1:10, 1:40, 1:160, and 1:640). An equal volume of dengue virus diluted to contain about 40-60 pfu is added to each serum dilution tube. Following incubation at 37° C. for 60 minutes, 0.2 mL are removed from each tube and inoculated onto triplicate wells of confluent LLC-MK2 in a 6-well plate. Each well is incubated at 37° C. for 90 minutes and the monolayers are then overlaid with 4 mL of 1% Carboxy Methyl Cellulose/Earle's Modified Medium. Plates are incubated for 7 days at 37° C. under 5% CO₂. Plaques are then counted, and the PRNT₅₀ is determined by using log probit paper. The percent reduction of plaques at each dilution level is plotted to determine the 50% reduction titer: plaque reduction points between 15% and 85% are used. Results are expressed as reciprocal of dilution. A serum is considered to be positive for the presence of neutralizing antibodies when the neutralizing antibodies titer thus determined is at least superior or equal to 1:10.

Response 30 Days after Vaccination

On Day 31, seroconversion rates were high against dengue type 2 viruses in all of the groups vaccinated with ChimeriVax™-DEN2. Low seroconversion rates to heterologous DEN serotypes 1, 3, and 4 were observed in YF-naïve subjects inoculated with ChimeriVax™-DEN2 at high or low dose. Seroconversion rates to DEN1 were 23% and 23% in the 5.0 and 3.0 log₁₀ PFU dose groups, respectively (Table 2); to DEN3 15% and 23%, respectively; and to DEN4 0% and 0%, respectively. In contrast, 100% of YF-immune subjects inoculated with ChimeriVax™-DEN2 seroconverted to all heterologous DEN serotypes.

ChimeriVax™-DEN2 vaccine induced very low cross-neutralizing antibody titers to the heterologous serotypes 1, 3, and 4 in YF-naïve subjects (Table 3). Geometric mean neutralizing antibody titers against heterologous dengue serotypes at Day 31 were significantly higher in YF-immune subjects vaccinated with ChimeriVax™-DEN2 than in YF-naïve subjects. For DEN1, geometric mean antibody titers in YF-immune subjects and YF-naïve subjects vaccinated with either 5.0 or 3.0 log₁₀ PFU ChimeriVax™-DEN2 were 79 vs. 10 and 12, respectively (p<0.0001). Similarly, for DEN3, titers were 73 vs. 13 and 12 (p<0.0001) (Table 3). None of the YF-naïve subjects seroconverted to DEN4. The geometric mean neutralizing antibody titer to DEN4 in YF-immune subjects was 57.

TABLE 2
Seroconversion rate (%) by treatment group, day 31

| | | CV-DEN2 | CV-DEN2 | CV-DEN2 |
|---|---|---|---|---|
| | YF-VAX ® | 3.0 log₁₀ PFU | 5.0 log₁₀ PFU | 5.0 log₁₀ PFU |
| Virus used in neutralization | YF-naïve | YF-naïve | YF-naïve | YF-immune |
| test | N = 13 | N = 13 | N = 13 | N = 14 |
| DEN2: strain 16681 | 0% | 92.3% | 100% | 100% |
| DEN2: ChimeriVax ™-D2 | 0 | 100 | 100 | 100 |
| DEN2: strain PR-159 | 0 | 84.6 | 92.3 | 100 |
| DEN2: strain JaH | 0 | 92.3 | 92.3 | 100 |
| DEN1: strain 16007 | 0 | 23.1 | 23.1 | 100 |
| DEN3: strain 16562 | 0 | 23.1 | 15.4 | 100 |
| DEN4: strain 1036 | 0 | 0 | 0 | 100 |

TABLE 3
Geometric mean antibody titer by treatment group, day 31

| | | CV-DEN2 | CV-DEN2 | CV-DEN2 |
|---|---|---|---|---|
| | YF-VAX ® | 3.0 log₁₀ PFU | 5.0 log₁₀ PFU | 5.0 log₁₀ PFU |
| Virus used in neutralization | YF-naïve | YF-naïve | YF-naïve | YF-immune |
| test | N = 13 | N = 13 | N = 13 | N = 14 |
| DEN2: strain 16681 | <10 | 365.0 | 358.6 | 383.3 |
| DEN2: ChimeriVax ™-D2 | <10 | 570.0 | 921.3 | 975.4 |
| DEN2: strain PR-159 | <10 | 313.8 | 218.3 | 724.5 |
| DEN2: strain JaH | <10 | 227.8 | 240.3 | 463.9 |
| DEN1: strain 16007 | <10 | 12.0 | 10.1 | 79.2 |
| DEN3: strain 16562 | <10 | 11.8 | 13.2 | 73.2 |
| DEN4: strain 1036 | <10 | <10 | <10 | 57.3 |

Response 6 Months after Vaccination

Response at 6 months after vaccination is given in Tables 4 and 5, below. At 6 months, low seropositivity rates to heterologous DEN serotypes 1, 3, and 4 were observed in YF-naïve subjects inoculated with ChimeriVax™-DEN2 at high or low dose. Seroconversion rates to DEN1 were 23% and 31% in the 5.0 and 3.0 log₁₀ PFU dose groups, respectively (Table 4); to DEN3 15% and 23%, respectively; and to DEN4 8% and 8%, respectively. In contrast, 100% of YF-immune subjects inoculated with ChimeriVax™-DEN2 were seropositive to DEN 1 and 3, and 64% to DEN4.

ChimeriVax™-DEN2 vaccine induced low cross-reactive neutralizing antibody titers to the heterologous serotypes 1, 3, and 4 in YF-naïve subjects (Table 5). Geometric mean neutralizing antibody titers against heterologous dengue serotypes at 6 months were higher in YF-immune subjects vaccinated with ChimeriVax™-DEN2 than in YF-naïve subjects. For DEN1, geometric mean antibody titers in YF-immune subjects and YF-naïve subjects vaccinated with either 5.0 or 3.0 log₁₀ PFU ChimeriVax™-DEN2, were 285 vs. <10 and 14, respectively. Similarly, for DEN3, titers were 268 vs. <10 and <10 (Table 5).

TABLE 4
Proportion seropositive (%) by treatment group, 6 months

| | | CV-DEN2 | CV-DEN2 | CV-DEN2 |
|---|---|---|---|---|
| | YF-VAX ® | 3.0 log₁₀ PFU | 5.0 log₁₀ PFU | 5.0 log₁₀ PFU |
| Virus used in neutralization | YF-naïve | YF-naïve | YF-naïve | YF-immune |
| test | N = 13 | N = 13 | N = 13 | N = 14 |
| DEN2: strain 16681 | 0% | 100% | 100% | 100 |
| DEN2: ChimeriVax ™-D2 | 0 | 100 | 100 | 100 |
| DEN2: strain PR-159 | 0 | 84.6 | 76.9 | 92.9 |
| DEN2: strain JaH | 0 | 76.9 | 69.2 | 92.9 |
| DEN1: strain 16007 | 0 | 30.8 | 23.1 | 100 |
| DEN3: strain 16562 | 0 | 23.1 | 15.4 | 100 |
| DEN4: strain 1036 | 0 | 7.7 | 7.7 | 64.3 |

TABLE 5
Geometric mean antibody titer by treatment group, 6 months

| | | CV-DEN2 | CV-DEN2 | CV-DEN2 |
|---|---|---|---|---|
| | YF-VAX ® | 3.0 log₁₀ PFU | 5.0 log₁₀ PFU | 5.0 log₁₀ PFU |
| Virus used in neutralization | YF-naïve | YF-naïve | YF-naïve | YF-immune |
| test | N = 13 | N = 13 | N = 13 | N = 14 |
| DEN2: strain 16681 | <10 | 568.6 | 285.1 | 870.2 |
| DEN2: ChimeriVax ™-D2 | <10 | 606.8 | 303 | 672 |
| DEN2: strain PR-159 | <10 | 55.1 | 49.5 | 160 |
| DEN2: strain JaH | <10 | 29.0 | 24.7 | 72.5 |
| DEN1: strain 16007 | <10 | 14.4 | <10 | 285.1 |
| DEN3: strain 16562 | <10 | <10 | <10 | 268.1 |
| DEN4: strain 1036 | <10 | <10 | <10 | 23.8 |

Response 1 Year after Vaccination

At 12 months, seropositivity rates were highest against dengue type 2 viruses in the YF-immune group vaccinated with ChimeriVax™-DEN2. This was particularly evident when the two DEN2 strains PR-159 and JAH were considered. These two strains are from the Americas and belong to two distinct variant groups (America I and II, respectively).

Low seropositivity rates to heterologous DEN serotypes 1, 3, and 4 were observed in YF-naïve subjects inoculated with ChimeriVax™-DEN2 at high or low dose. Seroconversion rates to DEN1 were 23% and 31% in the 5.0 and 3.0 log₁₀ PFU dose groups, respectively (Table 6); to DEN3 8% and 23%, respectively; and to DEN4 8% and 0%, respectively. In contrast, 100% of YF-immune subjects inoculated with ChimeriVax™-DEN2 were seropositive to DEN 1 and 3, and 29% to DEN4.

ChimeriVax™-DEN2 vaccine induced low cross-reactive neutralizing antibody titers to the DEN2 strains JaH and PR-159 and to heterologous serotypes 1, 3, and 4 in YF-naïve subjects (Table 7). Geometric mean neutralizing antibody titers against heterologous dengue serotypes at 12 months were significantly higher in YF-immune subjects vaccinated with ChimeriVax™-DEN2 than in YF-naïve subjects. For DEN1, geometric mean antibody titers in YF-immune subjects and YF-naïve subjects vaccinated with either 5.0 or 3.0 log₁₀ PFU ChimeriVax™-DEN2, were 89 vs. 10 and 13, respectively (p<0.0001). Similarly, for DEN3, titers were 72 vs. <10 and <10 (p<0.0001) (Table 7).

TABLE 6
Proportion seropositive (%) by treatment group, 12 months

| | | CV-DEN2 | CV-DEN2 | CV-DEN2 |
|---|---|---|---|---|
| | YF-VAX ® | 3.0 log₁₀ PFU | 5.0 log₁₀ PFU | 5.0 log₁₀ PFU |
| Virus used in neutralization | YF-naïve | YF-naïve | YF-naïve | YF-immune |
| test | N = 13 | N = 13 | N = 13 | N = 14 |
| DEN2: strain 16681 | 0% | 100% | 100% | 100 |
| DEN2: ChimeriVax ™-D2 | 0 | 100 | 100 | 100 |
| DEN2: strain PR-159 | 0 | 69.2 | 69.2 | 92.9 |
| DEN2: strain JaH | 0 | 61.5 | 53.8 | 85.7 |
| DEN1: strain 16007 | 0 | 30.8 | 23.1 | 100 |
| DEN3: strain 16562 | 0 | 23.1 | 7.7 | 100 |
| DEN4: strain 1036 | 0 | 0 | 7.7 | 28.6 |

TABLE 7
Geometric mean antibody titer by treatment group, 12 months

| | | CV-DEN2 | CV-DEN2 | CV-DEN2 |
|---|---|---|---|---|
| | YF-VAX ® | 3.0 log₁₀ PFU | 5.0 log₁₀ PFU | 5.0 log₁₀ PFU |
| Virus used in neutralization | YF-naïve | YF-naïve | YF-naïve | YF-immune |
| test | N = 13 | N = 13 | N = 13 | N = 14 |
| DEN2: strain 16681 | <10 | 368.9 | 183.3 | 744.1 |
| DEN2: ChimeriVax ™-D2 | <10 | 272.7 | 272.7 | 320.0 |
| DEN2: strain PR-159 | <10 | 42.2 | 30.6 | 72.5 |
| DEN2: strain JaH | <10 | 18.0 | 14.5 | 32.8 |
| DEN1: strain 16007 | <10 | 13.1 | 10.1 | 89.2 |
| DEN3: strain 16562 | <10 | <10 | <10 | 71.8 |
| DEN4: strain 1036 | <10 | <10 | <10 | <10 |

### Yellow Fever Antibody Response

Surprisingly, of the 14 YF immune subjects who were inoculated with ChimeriVax™-DEN2, only 2 (14%) had a boost in YF antibody. Thus, while preexisting YF immunity boosted the response to dengue serotypes 1-4 after ChimeriVax™ vaccination, the reciprocal was not true (i.e., ChimeriVax™-DEN2 did not boost antibody to yellow fever virus). This result is unexpected, since the mechanism underlying the broadened antibody response to dengue in yellow fever immune patients who received ChimeriVax™-DEN2 (shared epitopes between yellow fever and dengue envelope proteins) would have been expected to result in a boost in yellow fever antibodies following ChimeriVax™-D2. The results illustrate the unpredictability of cross-protective immune responses to flaviviruses and underline the novelty of the present invention.

### T Cell Responses

T cell responses were evaluated by IFNγ production in response to viral antigen in culture supernatants. Subjects were screened with inactivated viral cell lysate, which has been shown to generate primarily CD4+ T cell responses to the vaccine, but some CD8+ cells are also produced (Mangada et al., J. Immunol. Methods 284:89-97, 2004).

### Materials and Experimental Procedures

The T cell response was evaluated on Days 1 and 31 by measuring the production of IFNγ by PBMC stimulated in culture with inactivated virus antigen. Whole blood was collected on Days 1 and 31 in Vacutainer cell preparation tubes (CPT, BDBiosciences) and sent to Acambis, Inc. for isolation and cryopreservation of PBMC. Cells were washed in RPMI 1640, cryopreserved in heat-inactivated human AB serum (SeraCare, Oceanside Calif.) containing 10% DMSO, stored in liquid nitrogen, and thawed immediately before testing. For measuring IFNγ production, PBMC were cultured in 96-well flat bottom plates at 1.5×10⁵ cells per well for 7 days at 37° C. with 3 different glutaraldehyde-inactivated virus cell antigens: (1) ChimeriVax™-DEN2 virus (grown in Vero cells), (2) dengue 2 strain PUO218 virus (wild type dengue 2 virus grown in C6/36 cells), and (3) YF virus (grown in Vero cells). Controls consisted of inactivated mock-infected Vero or C6/36 cells. Inactivated viral antigen or control cell antigen was added at a concentration of 1:100 (15). PBMC were also stimulated with 1 μg/ml ConA as an assay positive control. IFNγ production was determined by ELISA using culture supernatants collected on Day 7.

### IFNγELISA

Culture supernatants were analyzed for IFNγ content by an indirect ELISA assay (OptEIA™ human IFNγ Kit, BDBiosciences-Pharmingen, Cat # 555142) according to the manufacturer's instructions.

### IFNγ Cytokine Production

IFNγ cytokine production was compared at Day 1 and Day 31 of the study (before vaccination and at Day 30 after vaccination) by testing the response to inactivated virus antigens. The administered vaccine (ChimeriVax™-DEN2), the parent wild type dengue-2 virus (PUO218), and the administered control virus (YF-VAX®) were tested. ChimeriVax™-DEN2 grown in Vero cells had very low background at Day 0, while dengue-2 virus grown in C6/36 cells produced responses in some of the subjects. Nonetheless, both of these antigens increased IFNγ production in each of the four vaccine groups. The inactivated YF-VAX® was not very immunogenic in any of the subjects, but it did show an increase at Day 31 relative to Day 1, especially in the YF vaccinated subjects.

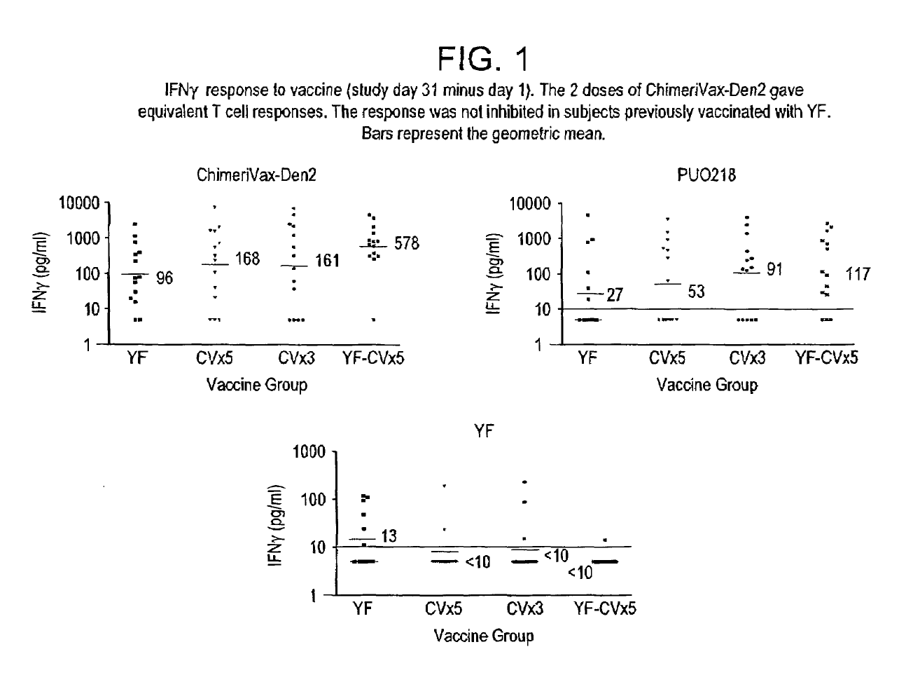

Comparisons between vaccination groups were made using the difference between values at Day 31 and Day 1 (FIG. 1). All groups responded to each of the inactivated antigens. Subjects who received 10³ or 10⁵ PFU of ChimeriVax™-DEN2 vaccine had equivalent IFNγ levels. In the IFNγ ELISA assay, ChimeriVax™-Den2 vaccinated subjects had slightly greater responses than YF vaccinated subjects (not significant) (FIG. 1).

Subjects who were YF pre-immune had an increase in the number of responders and an increase in the mean IFNγ level (FIG. 1). Table 8 summarizes the results showing the number of responders as a fraction of the total. About 65% of the ChimeriVax™-DEN2 and YF vaccinated subjects had a positive IFNγ response to the administered vaccine as test antigen, whereas approximately 90% of YF pre-immune subjects vaccinated with ChimeriVax™-DEN2 had a positive response (Table 8).

TABLE 8
Number of Subjects who responded to vaccine based on IFNγ response

| Immunization Group | CVD2 | Den2 PUO218 | YF |
|---|---|---|---|
| CVD2 10³ | 9/14 | 8/14 | 2/14 |
| CVD2 10⁵ | 9/14 | 7/14 | 1/14 |
| YF 10⁵ | 9/14 | 4/14 | 3/14 |
| YF-CVD2 10⁵ | 13/14 | 7/14 | 0/14 |

Positive response defined as 5-fold background, or ≧50 pg/ml at day 30 if Day 1 is less than 10 pg/ml (below sensitivity).

The results of this study show that approximately 65% of the ChimeriVax™-DEN2 and YF vaccinated subjects had a positive T cell response to the administered vaccine as test antigen, whereas ˜90% of YF pre-immune subjects vaccinated with ChimeriVax™-DEN2 had positive responses (defined by an IFNγ response). IFNγ production was the greatest in response to, ChimeriVax™-DEN2 vaccine virus.

The T cell responses in this clinical trial were consistent with the neutralizing antibody responses, in that both doses of vaccine stimulated similar T cell immune responses, and prior immunity to yellow fever virus did not inhibit the T cell response to ChimeriVax™-DEN2. The IFNγ responses were virtually the same for the 2 doses of ChimeriVax™-DEN2 (103 and 10⁵ pfu). The IFNγ response to ChimeriVax™-DEN2 was not diminished by prior vaccination with yellow fever virus and even higher numbers of responders were seen, suggesting a trend for enhanced T cell immunity in YF pre-immune subjects.

The inactivated antigen used in the assay identified the strongest responders but did not determine the specific proteins against which the immune response was generated. Inasmuch as an inactivated dengue antigen has been used, it is probable that primarily CD4+ responses are measured.

### Example 2

### Tetravalent Dengue Vaccine

### Study Design and Methods

In this Example, we evaluate in human subjects the immune response to sequential immunization with yellow fever 17D vaccine followed by administration of a mixture of four (4) chimeric yellow fever viruses that each comprise membrane and envelope proteins from dengue serotypes that are different from each other (ChimeriVax™-DEN tetravalent). The first stage of the study was to assess safety, tolerability, and immunogenicity of tetravalent ChimeriVax™-DEN vaccine containing serotypes DEN 1, 2, 3, and 4 in comparison to a yellow fever (YF) vaccine (YF-VAX®) and a placebo. The second stage of the trial evaluated safety and immunogenicity of sequential administration of YF-VAX®/tetravalent ChimeriVax™-DEN versus two doses of tetravalent ChimeriVax™-DEN given at a 5-9 month interval.

The study consisted of a screening period of 3 to 21 days before first vaccination, a double blind treatment period after first vaccination of 1 month, and a second 3 to 21 day screening period, before an open-label treatment period of 30 days commencing 5 to 9 months after first vaccination. A follow up visit at 12 months was planned.

In the first stage of the study, 3 groups of 33 healthy adult male and female subjects received a vaccination of tetravalent ChimeriVax™-DEN (group 1), YF-VAX® (group 2), or placebo (YF-VAX diluent—group 3), for a total of 99 subjects.

Prior to conducting any study-related procedures, subjects provided written informed consent. During screening, eligibility was assessed by a medical history, a physical examination, vital signs, clinical chemistry, hematology and serology (including serum pregnancy in female subjects), and a urine sample for urinalysis. On Day 1, subjects received a double-blind subcutaneous vaccination in the deltoid area and then attended the clinic on Days 3, 5, 7, 9, 11, 13, 15, 17, 19, and 21 for AE interview and blood sample collection for viremia. In addition, on Days 5, 9, 11, and 15, subjects provided a blood sample for clinical laboratory assessments. On Days 11 and 31, and at 5-9 months, subjects provided a blood sample for antibody analysis.

At 5 to 9 months, continued eligibility was assessed and an interim medical history recorded. Eligible subjects received a second vaccination (tetravalent ChimeriVax™-DEN vaccine) subcutaneously in the deltoid area. Subjects attended the clinic 2, 4, 6, 8, 10, 12, 14, 16, 18, and 20 days later for AE interview and blood sample collection for viremia. Blood samples for antibody tests were obtained 10 and 30 days after this second vaccination.

All subjects returned to the clinic 12 months after the initial vaccination (3-7 months after the second vaccination) for antibody tests. The design of the study is shown in Tables 9 and 10.

TABLE 9
Treatments to be administered at Day 1

| | | ChimeriVax ™- | YF-VAX ® | |
|---|---|---|---|---|
| | | DEN tetravalent | log₁₀ | |
| Group | Placebo | log₁₀ TCID50/dose | PFU/dose | N |
| 1 | — | ~4 ea. component | — | 33 |
| 2 | — | — | >4.74 | 33 |
| 3 | 0.5 mL | — | — | 33 |

TABLE 10
Treatments to be administered at 5 to 9 Months

| | ChimeriVax ™- | |
|---|---|---|
| | DEN tetravalent | |
| | log₁₀ | |
| Group | TCID50/dose | N |
| 1 | ~4 ea. | 33 |
| | component | |
| 2 | — | 33 |
| 3 | — | 33 |

The accurate Tetravalent ChimeriVax™-DEN dose per serotype is determined by TCID₅₀ assay as being 3.7/3.1/3.8/3.2 TCID50 for serotype 1, 2, 3, and 4 respectively.

Criteria for evaluation of immune responses were as follows:

The primary endpoint for immunogenicity is the seroconversion rate to dengue serotypes 1-4 at Day 31, using constant-virus, serum-dilution 50% plaque-reduction neutralization test (PRNT₅₀) performed as described in Example 1. This analysis defines seroconversion rates to all four dengue serotypes and to each individual serotype. Subjects that are seronegative at baseline (<1:10) will require a PRNT₅₀ titer of ≧1:10 to meet the criteria for seroconversion.

Secondary endpoints included the analysis of geometric mean neutralizing antibody titer to each dengue serotype and seroconversion rate 5 to 9 months after the first vaccination and 12 months after the first vaccination (i.e., 3-7 months after the second, booster vaccination). These serological responses are compared for subjects who received (a) a single dose of ChimeriVax™-DEN tetravalent; (b) two doses of ChimeriVax™-DEN tetravalent, or (c) a dose of yellow fever 17D vaccine (YF-VAX®) followed by 1 dose of ChimeriVax™-DEN tetravalent administered 5-9 months later.

### Results

The objective of the study was to evaluate the breadth of the immune response across all 4 dengue serotypes following different immunization regimes. The goal of immunizing human subjects against dengue virus disease is to achieve as broad a cross-neutralizing antibody response as possible. The immune responses of all subjects (which were dengue and yellow fever-naïve at baseline) 30 days after the second dose of study medication are shown in Table 11 (results against wild type strains).

54 subjects (who were dengue and yellow fever-naïve at baseline) received a single dose of ChimeriVax™-DEN tetravalent at Day 1 or placebo on day 1 followed by a single dose of ChimeriVax™-DEN tetravalent at 5-9 months. The neutralizing antibody responses 30 days after receipt of the active vaccine in these groups are combined and are shown in the far right-hand column of Table 11. A minority of subjects who received a single inoculation of ChimeriVax™-DEN tetravalent had a cross-reactive immune response to 3 or 4 dengue serotypes. Only 43% and 17% of subjects receiving one dose of ChimeriVax™-DEN tetravalent developed neutralizing antibodies to at least 3 or 4 dengue serotypes, respectively.

27 subjects (who were dengue and yellow fever-naïve at baseline) received 2 doses of ChimeriVax™-DEN tetravalent on Day 1 and at 5-9 months (Group 1). The neutralizing antibody responses are shown in Table 11. The breadth of the neutralizing antibody response in Group 1 was greater than in subjects who received only one dose of ChimeriVax™-DEN tetravalent. 55.6% and 40.7% of subjects receiving two doses of ChimeriVax™-DEN tetravalent developed neutralizing antibodies to 3 or 4 dengue serotypes, respectively.

26 subjects (who were dengue and yellow fever-naïve at baseline) received yellow fever vaccine (YF-VAX®) on Day 1 followed by one dose of ChimeriVax™-DEN tetravalent at 5-9 months (Group 2). The neutralizing antibody responses in Group 2 are shown in Table 11. The breadth of the neutralizing antibody response in Group 2 was greater than that in subjects who received either 1 dose of ChimeriVax™-DEN tetravalent or two doses of ChimeriVax™-DEN tetravalent separated by 5-9 months, 92% and 65% of subjects receiving sequential immunization with yellow fever vaccine and ChimeriVax™-DEN tetravalent vaccine developed neutralizing antibodies to at least 3 or 4 dengue serotypes, respectively.

The results clearly show that a sequential immunization regime in which yellow fever vaccine is given before a ChimeriVax™-DEN tetravalent vaccine results in a superior immune response to dengue with broad cross-reactivity across the dengue serotypes, than can be achieved with one or two doses of tested ChimeriVax™-DEN tetravalent vaccine alone.

Although the foregoing invention has been described in some detail by way of illustration and example for purposes of clarity of understanding, it will be readily apparent to those of ordinary skill in the art in the light of the teachings of this invention that certain changes and modifications may be made thereto without departing from the spirit or scope of the appended claims. All references cited above are incorporated herein by reference.

TABLE 11
Open-Label Treatment Period
Antibody Response Before Vaccination and at 10 and 30 Days After the second vaccination
(administered 5-9 months after the primary dose)
Against At Least One Serotype, At Least Two Serotypes, At Least Three Serotypes and to the Four Serotypes
of Dengue Strains (types 1-4), by Treatment Group

| | | | Group 1: | Group 2: | Pooled |
|---|---|---|---|---|---|
| | | | ChimeriVax-DEN -> | YF-VAX -> | Single Dose |
| | Time After | | ChimeriVax-DEN | ChimeriVax-DEN | ChimeriVax-DEN |
| | Vaccination[1] | Seropositive[2] | (N = 27) | (N = 26) | (N = 54) |
| At least one serotype | 0 days | Yes | 26 (96.3%) | 6 (23.1%) | 7 (13.0%) |
| | | No | 1 (3.7%) | 20 (76.9%) | 47 (87.0%) |
| | | Missing | 0 (0.0%) | 0 (0.0%) | 0 (0.0%) |
| | 10 days | Yes | 24 (88.9%) | 14 (53.8%) | 9 (16.7%) |
| | | No | 1 (3.7%) | 10 (38.5%) | 43 (79.6%) |
| | | Missing | 2 (7.4%) | 2 (7.7%) | 2 (3.7%) |
| | 30 days | Yes | 27 (100.0%) | 25 (96.2%) | 52 (96.3%) |
| | | No | 0 (0.0%) | 1 (3.8%) | 2 (3.7%) |
| | | Missing | 0 (0.0%) | 0 (0.0%) | 0 (0.0%) |
| At least two serotypes | 0 days | Yes | 18 (66.7%) | 3 (11.5%) | 1 (1.9%) |
| | | No | 9 (33.3%) | 23 (88.5%) | 53 (98.1%) |
| | | Missing | 0 (0.0%) | 0 (0.0%) | 0 (0.0%) |
| | 10 days | Yes | 21 (77.8%) | 3 (11.5%) | 4 (7.4%) |
| | | No | 4 (14.8%) | 21 (80.8%) | 48 (88.9%) |
| | | Missing | 2 (7.4%) | 2 (7.7%) | 2 (3.7%) |
| | 30 days | Yes | 23 (85.2%) | 24 (92.3%) | 42 (77.8%) |
| | | No | 4 (14.8%) | 2 (7.7%) | 12 (22.2%) |
| | | Missing | 0 (0.0%) | 0 (0.0%) | 0 (0.0%) |
| At least three serotypes | 0 days | Yes | 8 (29.6%) | 1 (3.8%) | 0 (0.0%) |
| | | No | 19 (70.4%) | 25 (96.2%) | 54 (100.0%) |
| | | Missing | 0 (0.0%) | 0 (0.0%) | 0 (0.0%) |
| | 10 days | Yes | 15 (55.6%) | 2 (7.7%) | 1 (1.9%) |
| | | No | 10 (37.0%) | 22 (84.6%) | 51 (94.4%) |
| | | Missing | 2 (7.4%) | 2 (7.7%) | 2 (3.7%) |
| | 30 days | Yes | 15 (55.6%) | 24 (92.3%) | 23 (42.6%) |
| | | No | 12 (44.4%) | 2 (7.7%) | 31 (57.4%) |
| | | Missing | 0 (0.0%) | 0 (0.0%) | 0 (0.0%) |
| All 4 serotypes | 0 days | Yes | 4 (14.8%) | 0 (0.0%) | 0 (0.0%) |
| | | No | 23 (85.2%) | 26 (100.0%) | 54 (100.0%) |
| | | Missing | 0 (0.0%) | 0 (0.0%) | 0 (0.0%) |
| | 10 days | Yes | 7 (25.9%) | 1 (3.8%) | 0 (0.0%) |
| | | No | 18 (66.7%) | 23 (88.5%) | 52 (96.3%) |
| | | Missing | 2 (7.4%) | 2 (7.7%) | 2 (3.7%) |
| | 30 days | Yes | 11 (40.7%) | 17 (65.4%) | 9 (16.7%) |
| | | No | 16 (59.3%) | 9 (34.6%) | 45 (83.3%) |
| | | Missing | 0 (0.0%) | 0 (0.0%) | 0 (0.0%) |

[1]Day 0 is the day at which the second vaccination was administered at 5-9 mo. The antibody measured at this time-point is the result of the first vaccination 5-9 mo. earlier
[2]Neutralizing antibody titer ≧10

What is claimed is:

1. A method of inducing a long-lasting, cross-neutralizing immune response to dengue viruses in a patient, the method comprising administering to the patient:
(i) one dose of a yellow fever virus vaccine, and
(ii) one dose of a chimeric flavivirus vaccine comprising at least one chimeric flavivirus comprising a yellow fever virus backbone in which the sequences encoding the envelope protein of the yellow fever virus have been replaced with sequences encoding the envelope protein of a dengue virus,
wherein the chimeric flavivirus vaccine is administered at least 30 days and up to 10 years after administration of the yellow fever vaccine.

2. The method of claim 1, wherein the chimeric flavivirus comprises a yellow fever virus backbone in which the sequences encoding the membrane and envelope proteins of the yellow fever virus have been replaced with the sequences encoding the membrane and envelope proteins of a dengue virus.

3. The method of claim 1, wherein the dengue envelope and/or dengue membrane proteins are shuffled proteins.

4. The method of claim 1, wherein the chimeric flavivirus vaccine is administered to the patient 30, 60, or 90 days after administration of the yellow fever vaccine.

5. The method of claim 1, wherein the chimeric flavivirus is composed of a YF17D virus backbone.

6. The method of claim 1, wherein the yellow fever virus vaccine comprises a YF17D vaccine strain 17D-204, 17D-213, or 17DD.

7. The method of claim 1, wherein one chimeric flavivirus comprises a yellow fever virus backbone in which the sequences encoding the membrane and envelope proteins of the yellow fever virus have been replaced with the sequences encoding the membrane and envelope proteins of a dengue serotype 1 virus.

8. The method of claim 1, wherein one chimeric flavivirus comprises a yellow fever virus backbone in which the sequences encoding the membrane and envelope proteins of the yellow fever virus have been replaced with the sequences encoding the membrane and envelope proteins of a dengue serotype 2 virus.

9. The method of claim 1, wherein one chimeric flavivirus comprises a yellow fever virus backbone in which the sequences encoding the membrane and envelope proteins of the yellow fever virus have been replaced with the sequences encoding the membrane and envelope proteins of a dengue serotype 3 virus.

10. The method of claim 1, wherein one chimeric flavivirus comprises a yellow fever virus backbone in which the sequences encoding the membrane and envelope proteins of the yellow fever virus have been replaced with the sequences encoding the membrane and envelope proteins of a dengue serotype 4 virus.

11. The method of claim 1, wherein the chimeric flavivirus vaccine is a monovalent vaccine.

12. The method of claim 1, wherein the chimeric flavivirus vaccine is a tetravalent vaccine.

13. The method of claim 1, further comprising administration of a booster dose of a chimeric flavivirus vaccine, as defined in (ii) of claim 1, 6 months to 10 years after the first dose of the chimeric flavivirus vaccine.

14. A kit comprising:
(i) a yellow fever virus vaccine, and
(ii) a chimeric flavivirus vaccine comprising at least one chimeric flavivirus comprising a yellow fever virus backbone in which the sequences encoding the envelope protein of the yellow fever virus have been replaced with the sequence encoding the envelope protein of a dengue virus.

15. The kit of claim 14, wherein the chimeric flavivirus comprises a yellow fever virus backbone in which the sequences encoding the membrane and envelope proteins of the yellow fever virus have been replaced with the sequences encoding the membrane and envelope proteins of a dengue virus.

16. The kit of claim 14, wherein the dengue envelope and/or dengue membrane proteins are shuffled proteins.

17. The kit of claim 14, wherein the yellow fever virus vaccine comprises a YF17D strain.

18. The kit of claim 14, wherein the chimeric flavivirus is composed of a YF17D virus backbone.

19. The kit of claim 14, wherein the chimeric flavivirus vaccine comprises one chimeric flavivirus comprising a yellow fever virus backbone in which the sequences encoding the membrane and envelope proteins of the yellow fever virus have been replaced with the sequences encoding the membrane and envelope proteins of a dengue serotype 1 virus.

20. The kit of claim 14, wherein the chimeric flavivirus vaccine comprises one chimeric flavivirus comprising a yellow fever virus backbone in which the sequences encoding the membrane and envelope proteins of the yellow fever virus have been replaced with the sequences encoding the membrane and envelope proteins of a dengue serotype 2 virus.

21. The kit of claim 14, wherein the chimeric flavivirus vaccine comprises one chimeric flavivirus comprising a yellow fever virus backbone in which the sequences encoding the membrane and envelope proteins of the yellow fever virus have been replaced with the sequences encoding the membrane and envelope proteins of a dengue serotype 3 virus.

22. The kit of claim 14, wherein the chimeric flavivirus vaccine comprises one chimeric flavivirus comprising a yellow fever virus backbone in which the sequences encoding the membrane and envelope proteins of the yellow fever virus have been replaced with the sequences encoding the membrane and envelope proteins of a dengue serotype 4 virus.

23. The kit of claim 14, further comprising at least one booster dose of a chimeric flavivirus vaccine.

24. (canceled)
